(19) United States
(12) Patent Application Publication
(10) Pub. No.: US 2004/0249390 A1
(43) Pub. Date: Dec. 9, 2004

(54) Manual depilatory apparatus and method of use

(76) Inventors: Guita Dovas, New York, NY (US); Sharzad F. Boyd, New York, NY (US)

Correspondence Address: ABELMAN, FRAYNE & SCHWAB, 150 East 42nd Street, New York, NY, 10017-5612, US

(21) Appl. No.: 10/454,250

(22) Filed: Jun. 3, 2003

(51) Int. Cl. A61B 17/50

(52) U.S. Cl. 606/133

(57) ABSTRACT

An apparatus for removing hair from a body part is disclosed, which includes a spiral shaped wire of predetermined length and having a gripping handle fixedly attached to each end. When the coiled wire is pressed against an arcuate surface of skin to be depilated, and the handles maintained in generally parallel relation and rotated in opposite directions, the coils of wire rotate toward and away from the skin surface and progressively open and close their relative spacing to grip and remove upstanding hairs from their follicles in the skin. A method of removing hairs from human skin is also disclosed.

### BACKGROUND OF THE INVENTION

1. Field of the Invention

The invention relates to a manually operable device for removing hair from the face and other areas of the human body and a method of using same.

2. Description of the Related Art

Various depilatory devices have been developed for removing hair from the face, legs and other areas of the human body. One prior art device included a power-driven length of a tightly coiled wire spiral that was caused to rotate around its longitudinal axis. When the rotating coil was brought into contact with hair extending a sufficient distance from the skin, the free end of the hair was squeezed between the adjacent spiral sections of the moving wire and the rotation caused the entrained hair to be pulled free of the follicle. These electrically-powered devices are relatively expensive due to the complexity of their construction. These electrically-powered devices of the prior art are also bulky and not readily portable.

The traditional alternative for removing hair is a pair of cosmetic tweezers, which are specifically designed for this purpose. However, the use of tweezers generally requires a strong light and a mirror, preferably a magnifying mirror to facilitate the identification of the hair to be removed. The use of tweezers can be time consuming if more than one or two hairs are to be removed.

It is therefore an object of this invention to provide a small, lightweight, portable and reliable manual depilatory apparatus for removing unwanted body hair, sometimes referred to herein as hair strands.

It is a further object of this invention to provide such an apparatus that can be brought into contact with a portion of skin for removal of one or more individual hairs that does not require the user to identify and isolate individual strands of hair to be removed by the device.

It is still a further object of this invention to provide a depilatory apparatus for removing hair without the need of assistance of a mirror.

### SUMMARY OF THE INVENTION

The above objects and other advantages are obtained from the apparatus of the present invention which comprises a tightly-wound spiral of coiled wire, the opposing free ends of which are fitted with handles affixed thereto which permit the user to freely rotate the wire when its outer surface is moved in contact with human skin. The wire spiral assumes a linear, cylindrical configuration in an unstressed state, but is sufficiently flexible to readily assume an arcuate configuration when the opposing handles are moved into generally parallel relation adjacent each other.

In the method of use of the apparatus, each of the handles are held between the thumb and fingers of the user's right and left hand, and the spiral wound wire is moved from a straight configuration to an arcuate configuration by moving the handles toward each other. The arcuate portion of the wire is then pressed against an area of skin that is to be depilated and moved up and down while the fingers twist the handles in opposing rotational directions to cause the wire to rotate. The rotation causes the spacing between the coils of wires at the upper portion of the arc to increase while the portion of the coils inside the arc which contacts the skin are pushed together with sufficient force to retain any hairs that may have found their way between the adjacent spiral wraps so that they are pulled from the follicle.

### BRIEF DESCRIPTION OF THE DRAWINGS

The invention will be further described hereinbelow and with reference to the attached drawings, wherein:

FIG. 1 is plan view of the apparatus of the invention;

FIG. 2 is an enlarged cross-sectional view of one of the handles of the apparatus, taken along lines 2-2 of the handle of FIG. 1;

FIG. 3 is a plan view of an alternative embodiment the handles of the apparatus of FIG. 1, incorporating axially extending grooves to enhance gripping;

FIG. 4 is an enlarged plan view of the device of FIG. 1, illustrating one method of its use in removing facial hair from an arcuately shaped skin surface; and

FIG. 5 is a greatly enlarged cross-sectional view, taken along lines 5-5 of FIG. 4.

### DETAILED DESCRIPTION OF THE INVENTION

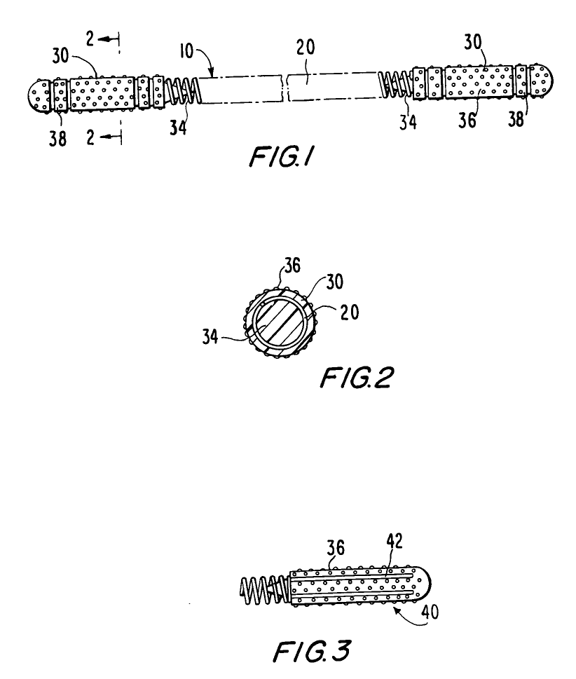

Referring to FIG. 1, the apparatus of the invention 10 is shown in an unstressed configuration where the tightly-wound spiral wire 20 is extended longitudinally and assumes a generally cylindrical configuration. Each end of the hollow cylindrical spiral is fitted with a handle 30 of a length and diameter that can be conveniently gripped between the user's thumb and fingers for rotation. As shown in the cross-sectional view of FIG. 2, the handles 30 can be provided with an internal cylindrically shaped opening 32 sized to receive the wire spiral in a tight-fitting and secured relation.

In the preferred embodiment of the invention shown in FIG. 2, the opening 32 also serves to define an interior post 34 that fits tightly within the hollow interior of spiral 20 to retain the spiral in fixed relation with each handle 30.

As will be apparent to one or ordinary skill in the art, other configurations and constructions, and means, such as adhesive, can be utilized to secure the handles 30 to each end of the spiral wound wire 20.

The handle 30 is provided with a plurality of small raised members 36 monolithically molded on the surface to improve the user's grip and to enhance the aesthetic appearance of the device. The handles are fabricated of molded plastic, but can also be made of wood or metal and formed by machining, molding or casting. In one preferred embodiment, the handles are completely formed as illustrated in FIGS. 1 and 2, of molded plastic. In addition, a plurality of circumferential grooves are provided on the handle to enhance the grip. Alternatively any appropriate surface roughening or texturing may be provided to enhance the grip.

FIG. 3 illustrates an alternative embodiment of a handle 40 of the apparatus, wherein elongated axially extending grooves 42 are provided to enhance the group. The handle 40 also includes appropriate surface roughening, preferably a plurality of small raised members 36 monolithically molded with the handle as disclosed in FIG. 1. As noted, any surface roughening can be used.

As will be understood by one of ordinary skill in the art, the invention provides a simple yet effective depilating device that is compact, lightweight, portable and inexpensive to manufacture. It can be made in various sizes to accommodate different parts of the body. It can be carried in a cosmetic bag, travel kit, or the like. It can be produced at low cost and is easy to use. It is relatively compact in size, and its portability will therefore permit users to keep separate devices at home, in the office and in a travel kit.

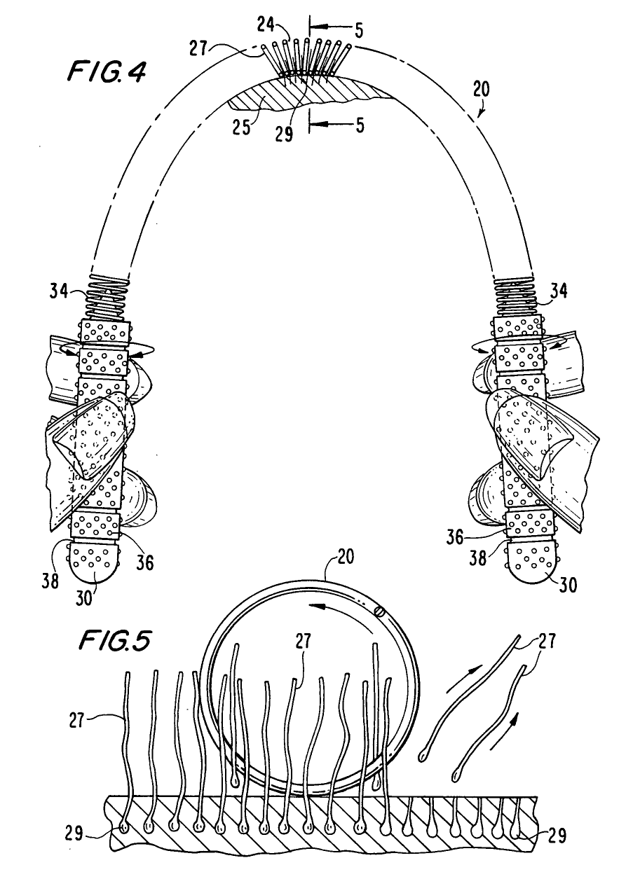

As illustrated in FIGS. 4 and 5, the hair removal apparatus of the invention is used by gripping the handles 30 between the thumb and fingers of each hand and bringing the handles into approximately parallel alignment. This causes the central portion 24 of the spiral wound wire 20 to assume an arcuate configuration. The arcuate portion 24 of the device is brought into moving contact with the area of skin to be depilated, while rotating the handles in opposite directions. This movement is thereby transmitted to spiral wound wire 20 which engages, grips and pulls any hairs from the skin as the device is moved along the surface. As the coils rotate along the skin surface, the spaces between the coils most distal from the skin gradually decrease as they rotate toward the skin surface while gripping upstanding hair strands 27. Continued rotation causes them to pull the hairs 27 out of their follicles 29. As the rotation continues the coils separate and repeat the process of progressively opening and closing while pulling the upstanding hairs from their roots.

As with any process of physically removing hair from its follicles, some discomfort can be expected. In the practice of the method of the invention, any discomfort can be minimized or eliminated by applying a skin-numbing lotion, cream or solution to the depilated area before use of the device. A soothing or cooling lotion or cream can be applied after the treatment to minimize any subsequent discomfort.

I claim:

1. Apparatus for removing hair from a body part, which comprises a spiral shaped wire of predetermined length and having a gripping handle attached to each end, such that rotation of each handle causes corresponding rotation of said spiral shaped wire.

2. The apparatus according to claim 1, wherein each said gripping handle defines at least one surface discontinuity to improve the grip thereof.

3. The apparatus according to claim 2, wherein said surface discontinuity comprises at least one groove.

4. The apparatus according to claim 3, wherein said surface discontinuity comprises a plurality of grooves.

5. The apparatus according to claim 4, wherein each said handle has a cylindrical shape, and each said groove is generally circumferential.

6. The apparatus according to claim 5, wherein each said handle is molded plastic and includes a plurality of monolithically formed raised members to enhance the user's grip on said handle.

7. The apparatus according to claim 3, wherein each said groove extends generally axially of said handle.

8. The apparatus according to claim 7, wherein each said handle includes a plurality of axially extending grooves.

9. The apparatus according to claim 1, wherein said spiral shaped wire is made of steel.

10. The apparatus according to claim 9, wherein said steel is stainless steel.

11. The apparatus according to claim 1, wherein each said handle defines a generally axially extending opening for reception of an end portion of said spiral shaped wire.

12. The apparatus according to claim 1, wherein said generally axially extending opening includes a post extending therethrough and inserted into each open end of said spiral shaped wire for attachment of each said handle to said spiral shaped wire.

13. A portable apparatus for removing hair from a body part, comprising a generally cylindrical tightly wound coiled flexible spiral shaped steel wire of predetermined length, a handle fixedly attached to each end of said spiral shaped steel wire, each handle having gripping means to enable a user to securely grip said handles to flex said wire to position said wire in engagement with the outer surface of a body part to remove hair therefrom, such that rotating said handles in opposite direction causes said coils of said wire to traverse the body part while progressively increasing and decreasing the spacing therebetween so as to grip hair strands and remove them from the body part.

14. A method of removing hair from a human body part utilizing a generally tightly wound coiled flexible spiral shaped steel wire of predetermined length and having a handle fixedly attached to each end, comprising:
a) gripping each of said handles and flexing said coiled wire to form an arcuate shape;
b) positioning said handles in generally parallel relation;
c) pressing an accurate portion of said coiled wire against the surface of the body part to be depilated and maintaining said handles in said generally parallel relation; and
d) manually rotating said handles in opposite directions while moving said spiral wound coiled wire across said surface thereby progressively decreasing the spaces between said coils as they rotate toward the skin surface, to grip upstanding hairs and remove them from their follicles.
